(19) United States
(12) Patent Application Publication
(10) Pub. No.: US 2008/0228072 A1
(43) Pub. Date: Sep. 18, 2008

(54) Foreign Body Identifier

(75) Inventors: Jeffrey H. Nycz, Collierville, TN (US); Carlos E. Gil, Collierville, TN (US); William T. Donofrio, Andover, MN (US); Stanley Warren Olson, Germantown, TN (US); Jeetendra Bharadwaj, Memphis, TN (US)

Correspondence Address: Haynes and Boone, LLP;Suite 3100, 901 Main Street, Dallas, TX, 75202-3789, US

(73) Assignee: WARSAW ORTHOPEDIC, INC., Warsaw, IN (US)

(21) Appl. No.: 11/687,574

(22) Filed: Mar. 16, 2007

(51) Int. Cl. A61B 8/00; A61B 17/00; A61B 17/08; A61B 5/05

(52) U.S. Cl. 600/437; 600/407; 606/1; 606/158

(57) ABSTRACT

A surgical instrument for the presence and/or location of a foreign body is disclosed. The surgical instrument is hand-held. In some embodiments, the surgical instrument includes transducers adapted for emitting and/or receiving signals. In such embodiments, the surgical instrument utilizes pulse-echo measurements to determine characteristics and/or location of the foreign body. In other embodiments, the surgical instrument includes a measurement circuit for detecting the presence and/or location of a foreign body by a change in the characteristics of the measurement circuit. The surgical instrument may be utilized to determine such things as the size of a foreign body, the orientation of a foreign body with respect to patient anatomy and/or another foreign body, and whether the foreign body has been completely removed.

### FIELD OF THE INVENTION

The present disclosure is directed to improved instrumentation for detecting the presence and/or location of a foreign body and methods of using such instrumentation. More particularly, in one aspect the present disclosure is directed toward medical instruments and methods for locating a foreign body within the tissue of a patient.

### BACKGROUND OF THE INVENTION

The location of foreign bodies within a patient is often necessary in emergency care situations. However, the typical imaging techniques for locating foreign bodies, such as radiographs, CT scans, or MRI scans, can be expensive, time-consuming, immobile, and inconvenient. Further, these imaging techniques may have difficulty locating certain types of materials.

Although the existing systems and methods have been generally adequate for their intended purposes, they have not been entirely satisfactory in all respects.

### SUMMARY

The present disclosure provides a surgical instrument that includes an energy source and a sensor for detecting reflected energy. A processor evaluates the reflected energy. In one aspect, the processor determines the presence of a foreign body. In another aspect, the processor determines the location of a foreign body.

In another aspect, the present disclosure provides a handheld surgical instrument for use in detecting a foreign body within a tissue. The surgical instrument includes a housing having an external gripping portion and a sensor portion having a conductive surface. The sensor portion is adapted to be in conductive contact with a surface of the tissue. The surgical instrument also includes an energy source adapted for emitting an energy signal into the tissue. The energy signal is configured to pass through the tissue and at least partially reflect off a boundary between the foreign body and the tissue. The surgical instrument also includes a sensor adapted for detecting the reflected signal and a processor for determining a characteristic of the foreign body based on the reflected signal.

In another aspect, the present disclosure provides a method of detecting a foreign body within a tissue. The method includes placing an energy transducer and a sensor in conductive contact with the tissue, where the energy transducer is adapted to emit energy signals and the sensor is adapted to receive reflected energy signals. The energy transducer and the sensor are positioned within a portable handheld device. The method also includes emitting an energy signal into the tissue, where the energy signal is adapted to pass through the tissue and at least partially reflect off a boundary of the foreign body within the tissue. The method also includes receiving at least a portion of the reflected signal at the sensor. The method also includes determining a characteristic of the foreign body based on the portion of the reflected signal received.

In another aspect, the present disclosure provides a method of removing a foreign body from a tissue. The method includes detecting the presence of the foreign body within the tissue by placing a hand-held device in conductive contact with the tissue. The hand-held device includes an energy source adapted for emitting an energy signal into the tissue, where the energy signal is configured to pass through the tissue and at least partially reflect off a boundary between the foreign body and the tissue. The hand-held device also includes a sensor adapted for detecting the reflected signal and a processor for determining a characteristic of the foreign body based on the reflected signal. The method also includes determining the location and volume of the foreign body using the hand-held device and utilizing the location and volume of the foreign body to guide a surgical instrument to the foreign body. Finally, the method includes removing the foreign body with the surgical instrument.

In another aspect, the present disclosure provides a handheld surgical instrument for use in detecting a foreign body within a tissue. The instrument includes a housing having a proximal portion, a distal portion, and a longitudinal axis extending therebetween. A gripping portion is positioned adjacent the proximal portion and is adapted for grasping by a user. A sensor circuit is positioned within the distal portion. The sensor circuit is configured such that a characteristic of the sensor circuit is modified by a proximity of the foreign body to the sensor circuit. The instrument also includes a processor for determining a position of the foreign body based on a value of the characteristic of the sensor circuit that is modified by the proximity of the foreign body to the sensor circuit.

In another aspect, the present disclosure provides a method of detecting a foreign body within a tissue. The method includes providing a hand-held device having a sensor circuit and a processor, where the sensor circuit is configured such that a characteristic of the sensor circuit is affected by a proximity of the foreign body to the sensor circuit. The processor determines a position of the foreign body based on the value of the characteristic of the sensor circuit affected by the proximity of the foreign body to the sensor circuit. The method also includes positioning the hand-held device adjacent the tissue and monitoring the characteristic of the sensor circuit affected by the proximity of the foreign body to the sensor circuit. The method also includes producing an alert in response to a change in the characteristic of the sensor circuit indicative of the presence of the foreign body. The method also includes determining the position of the detected foreign body within the tissue based on the value of the characteristic of the sensor circuit affected by the proximity of the foreign body to the sensor circuit.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1 is a front view of an electronic instrument for detecting a foreign body according to one embodiment of the present disclosure in use with a patient's hip region.

FIG. 2 is an enlarged front view of the electronic instrument of FIG. 1 in use with the patient's hip region.

FIG. 3 is a schematic illustration of an electronic instrument according to one embodiment of the present disclosure.

FIG. 4 is a perspective view of a signal of an electronic instrument having a fan shape according to one embodiment of the present disclosure.

FIG. 5 is a perspective view of a signal of an electronic instrument having a substantially conical shape according to one embodiment of the present disclosure.

FIG. 6 is a perspective view of a signal of an electronic instrument having a focused beam according to one embodiment of the present disclosure.

FIG. 7 is a partial cross-sectional view of a portion of an electronic instrument according to one embodiment of the present disclosure.

FIG. 8 is a schematic view of an instrument according to one embodiment of the present disclosure.

FIG. 9 is a partial, cutaway side view of an instrument according to one embodiment of the present disclosure.

FIG. 10 is a partial, cutaway side view of the instrument of FIG. 9 in an alternative position.

FIG. 11 is a schematic illustration of a circuit of an instrument according to one embodiment of the present disclosure.

FIG. 12 is a schematic view of an instrument according to one embodiment of the present disclosure.

FIG. 13 is a schematic view of an instrument according to one embodiment of the present disclosure.

FIG. 14 is a perspective view of an instrument according to one embodiment of the present disclosure.

FIG. 15 is a lateral view of the instrument of FIG. 14.

FIG. 16 is a top view of the instrument of FIG. 14.

FIG. 17 is a perspective cutaway view of the instrument of FIG. 14.

FIG. 18 is an exploded view of the instrument of FIG. 14.

FIG. 19 is a cross-sectional side view of the instrument of FIG. 14.

### DESCRIPTION OF THE PREFERRED EMBODIMENTS

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates.

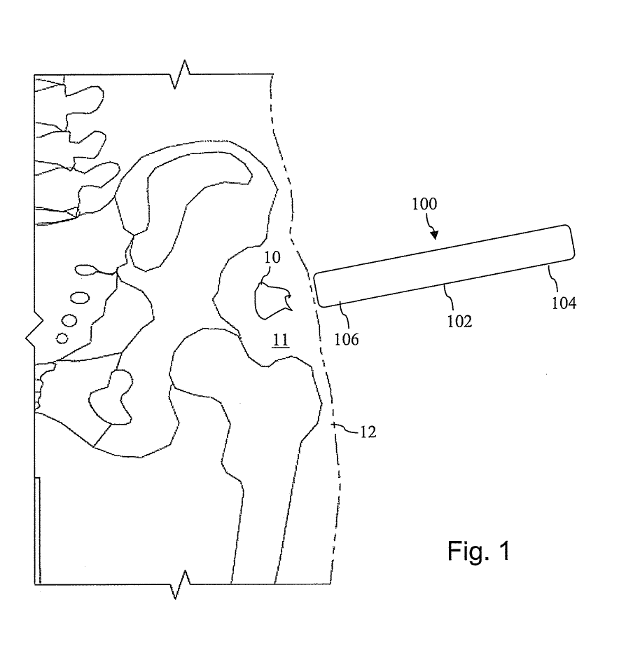

Referring now to FIG. 1, there is shown electronic instrumentation 100 for detecting a foreign body 10 within a tissue 11 of a patient according to one aspect of the present disclosure. In this regard, foreign body 10 includes any physical or chemical article not occurring normally found in a healthy tissue. For example, the foreign body 10 may be metallic, glass, plastic, or another material and may include such items as bullets, nails, glass fragments, shrapnel, other objects, and parts thereof. Further, the foreign body 10 may be a naturally occurring article such as a tumor, lesion, bone fragments, enzymes, or other medical condition that is not normally found in the healthy tissue being inspected. Also, the foreign body 10 is illustrated as single component. This is illustration purposes only. In many instances the foreign body 10 will be comprised of a plurality of pieces or fragments. The tissue 11 in FIG. 1 is generally represented as being soft tissue. However, the tissue 11 may be soft or hard tissue, including muscles, bone, ligaments, cartilage, and other tissues.

The electronic instrumentation 100 is generically pictured and includes a main body 102, a proximal portion 104, and a distal portion 106. The main body 102 and/or the proximal portion 104 may include a gripping surface for grasping by the user or engagement with a further instrument. The distal portion 106 is adapted for placement adjacent a surface 12 of the tissue 11 when the electronic instrumentation 100 is in use. As shown, the proximal portion 104 is disposed away from the tissue 11 when the electronic instrumentation 100 is in use. Though not explicitly shown in FIG. 1, the electronic instrumentation 100 may include additional features and components. For example, in some embodiments the electronic instrumentation 100 may include an output for communicating information to a user, such as a visual display, a speaker, tactile feedback, and/or a signal output (wired or wireless) for communication with another device. Further, the electronic instrumentation 100 may include components for providing positioning information, such as fiducial markers, accelerometers, gyroscopes, and/or other components for providing positional information. As described below, the additional components may be disposed within the main body 102 or external to the main body. Additional details about sensors and their use is found in U.S. patent application Ser. No. 11/356,643 filed Feb. 17, 2006 Surgical Instrument to Access Tissue Characteristics, which application is hereby incorporated by reference in its entirety.

The main body 102 is adapted for housing the various electronic components of the electronic instrumentation 100. In FIG. 1, the main body 102 is shown as being substantially cylindrical and elongated. This is merely for illustrative purposes. It is fully contemplated that the main body 102 may take any shape capable of holding the components of the electronic instrumentation 100, including non-cylindrical and non-elongated designs. However, it is preferred that the main body 102 be of appropriate shape and size to be portable and handheld. For example, but without limitation, the main body may be of similar design, shape, and size to an injector gun, laser pointer, or pen. Still further, in another embodiment the main body 102 is narrow like a catheter or needle and is manipulated remotely for minimally invasive surgery. In some embodiments, the housing may be substantially similar to the housing shown in FIGS. 14-19.

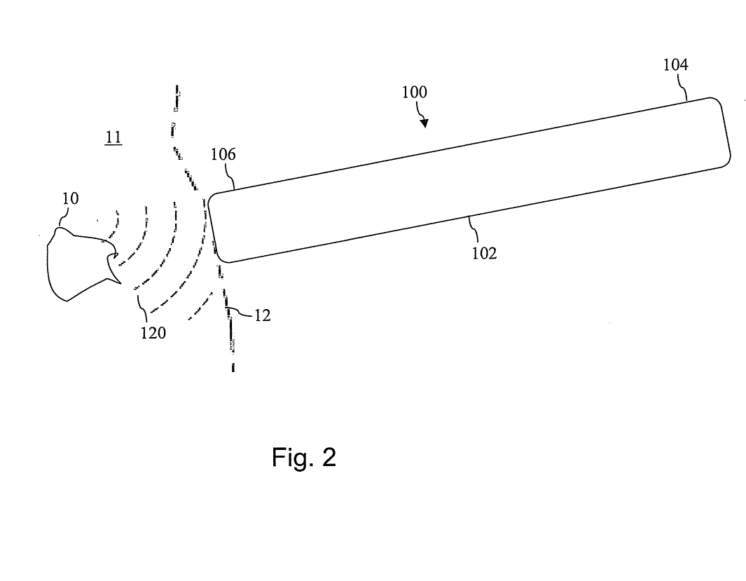

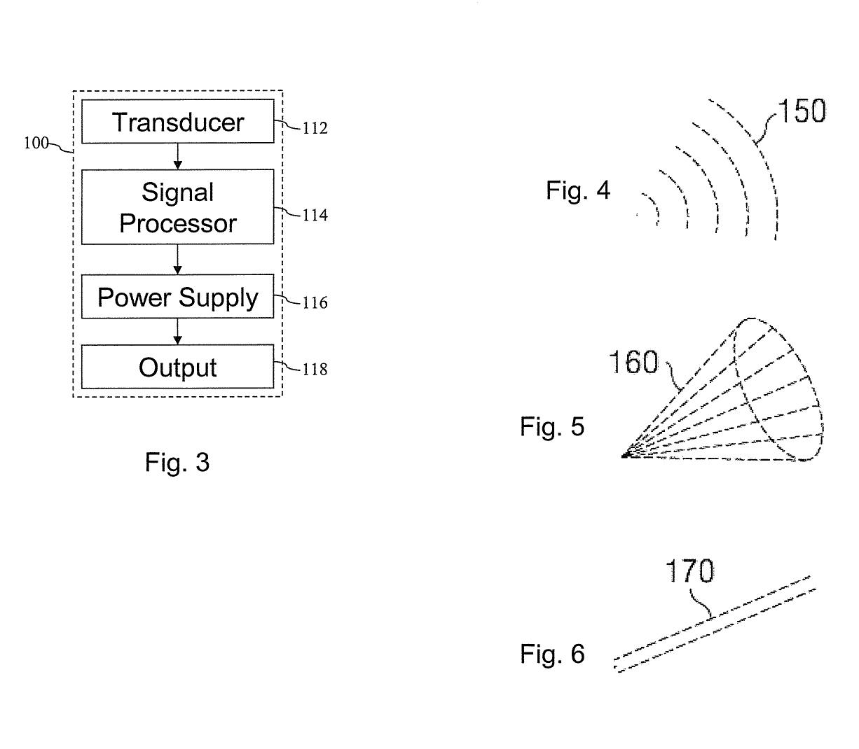

Referring now to FIGS. 2 and 3, the electronic instrumentation 100 will now be described in greater detail. FIG. 2 is a front view of the electronic instrumentation 100 in use detecting the foreign body 10 within the tissue 11. FIG. 3 is a schematic depiction of the electronic instrumentation 100. As shown in FIG. 3, the electronic instrumentation 100 includes at least an acoustic transducer 112, a signal processor 114, a power supply 116, and an output 118. The acoustic transducer 112 is adapted for emitting and detecting acoustic signals. In this regard it is contemplated that the acoustic transducer 112 may function as a pulse-echo transducer having a single element for emitting and receiving acoustic signals. In that regard, the acoustic transducer 112 may include an energy source for producing or emitting a signal 120 (FIG. 2) and a sensor for detecting an echo or reflection signal of the signal 120. The function of the energy source and the sensor may be performed by a single element or component switched between a transmit mode and a listen mode. On the other hand, the acoustic transducer 112 may be a dual-element transducer where a first element is configured for emitting the acoustic signal 120 and a second element is configured to receive or detect reflected acoustic signals. It is fully contemplated that the acoustic transducer 112 may be piezoelectric.

In some aspects, the acoustic transducers and acoustic signals of the present embodiment may be used in the frequency range of ultrasonic signals. In some high resolution systems of the present invention, the frequency can range from 20 KHz up to and exceeding 300 MHz. For example, these frequencies may be used in acoustic microscopic instrument applications. In one aspect of the present invention, the frequency range is between 1 MHz to 15 MHz. It is to be understood that acoustic signals are a form of transmitted energy. It is fully contemplated that in an alternative embodiment, the transducer(s) of the electronic instrumentation are adapted for use with other forms of energy and/or different frequencies. For example, in some embodiments lasers, visible light, radio frequency, microwaves, electromagnetic, magnetic, and other forms of energy may be utilized provided they can be transmitted into the tissue to detect the presence of a foreign body. For example, in some embodiments the energy source may utilize RF energy in the range from 400 KHz up to 10 GHz. In still further embodiments the energy source could utilize a light source for generating non-coherent and/or coherent (laser) light.

The acoustic transducer 112 is adapted for placement adjacent the distal portion 106 of the electronic instrumentation 100. In fact, the acoustic transducer 112 may itself substantially form the end of the distal portion 106. The acoustic transducer 112 is adapted for placement at the distal portion 106 such that when the electronic instrumentation 100 is in use the transducer can emit an acoustic signal or other type of energy wave into the tissue 11 and receive an echo or return signal from the tissue. The distal portion 106 may include a conductive surface. Conductive surface in this context does not require, but may include electrical conductivity. Rather, conductive surface in this context is intended to mean a surface configured to facilitate the emitting and receiving of the acoustic signals. Thus, the surface may serve as the transducer to emit or receive the signal, or the surface may simply be transmissive allowing the signals to pass through. Moreover, in one aspect the conductive surface is formed as a disposable sheath such that it is discarded after each use and the instrument housing with sensing hardware may be reused.

The strength and frequency of the acoustic signal can be varied depending on the type of tissue being evaluated and/or the type of foreign object being detected. Further, the strength and frequency of the signal may be varied to enhance the accuracy of evaluation of a boundary of the foreign object to assist in determining the location of the foreign object. For instance, the instrument may evaluate the tissue and foreign object with energy beams of multiple frequencies and then integrate the sensed information to best approximate the size, boundaries, and/or location of the foreign object. Further, the energy beam or signal may be shaped for optimum performance and may include a focused beam and/or a more diffuse beam projection. In some embodiments, the acoustic signal or other energy signal is emitted with a substantially cylindrical or conical shape.

Consider the case of the foreign body 10 within the tissue 11, as shown in FIGS. 1 and 2. The distal portion 106, including the acoustic transducer 112, is placed in conductive contact with at least the exterior surface 12 of the tissue 11. In this way conductive contact implies that the distal portion 106 or active end of the instrument 100 is in sufficient contact, either direct or indirect, with the tissue to emit an acoustic signal or beam into the tissue and receive a reflected acoustic signal from a boundary between the foreign body and the healthy tissue. In one aspect, the distal portion 106 is in direct contact with the surface 12 or in indirect contact via a coupling medium. In some embodiments, distal portion 106 of the electronic instrumentation 100 is formed of an appropriate shape and material to pierce through the surface 12 and into the tissue 11.

In one embodiment, the reflected signals are used to determine initial points or locations indicative of a boundary of the foreign body 10. The boundary points may be saved in a memory of the electronic instrumentation. The transducer 112 may then be moved to a different location with respect to the tissue 11 and foreign body 10, or the orientation of the transducer may be changed relative to the position from which the initial boundary points were determined. In the new position, a series of second group of points indicative of a boundary of the foreign body 10 may be calculated based on the reflected signals and saved in memory. These first and second points may then be combined and used to approximate the boundaries of the foreign body 10 and, in some embodiments, to approximate a volume or size of the foreign body. Further, in at least one approach, the points are compared to one or more known geometric shapes of known volume to determine the best fit and thereby determine the best approximation of the volume of the foreign body. For example, but without limitation to other shapes, the geometric shapes include spheres, cylinders, cubes, pyramids and cones. In addition, more than one shape of different sizes may be used to approximate the shape and/or volume of the foreign body. For example, a series of small cubes may be stacked in virtual space within the detected boundaries points to closely approximate the actual sensed volume.

The acoustic transducer 112 emits an acoustic signal 130 into the tissue 11 through exterior surface 12. The acoustic signal 130 will pass through the tissue 11 until it arrives at the interface between the foreign body and the tissue. At that point, a portion of the acoustic signal will reflect off of the foreign body 10. This reflection is the echo or return signal that will be received by the acoustic transducer 112. If no reflection is received, then the transducer 112 may not be in alignment with the foreign body. In that regard, the electronic instrumentation may be passed over the surface 12 of the tissue 11 until reflected signals indicative of the presence of the foreign body 10 are obtained. In one embodiment, based on the time delay of the return signal 132 and the assumed constant speed of the acoustic signal in the tissue 11, the depth of the location of the foreign body 10 may be determined by the signal processor 114. Further, receiving reflected signals from a variety of approaches or angles can allow the signal processor 114 to determine the 3-D position of the foreign body 10 within the tissue and/or more accurately determine the boundaries of the foreign body. Further, a plurality of depth readings may facilitate determining the size or volume of the foreign body 10. As described more fully below, the process for determining the size or volume of the foreign body 10 may range from a single pulse-echo reading, a plurality of pulse-echo readings, pulse-echo readings accompanied with position data, and/or other means of determining volume. It should be noted that determining volume is intended to include approximations and estimations of actual volume.

While in some embodiments the electronic instrumentation 100 may obtain useful data from a single pulse-echo reading, it is contemplated that the electronic instrumentation may be rotated about its longitudinal axis to sequentially assess the foreign body 10. In this respect, it is contemplated that the transducer 112 may be adapted to produce an acoustic beam with an appropriate shape for determining the presence and/or size of the foreign body 10. For example, as shown in FIG. 4 in some embodiments the signal beam 150 of the transducer 112 may be substantially fan shaped. Where the beam is fan shaped it may be adapted for detecting the foreign body 10 in only a single plane, corresponding to the plane of the beam. In this case, the electronic instrumentation 100 may be rotated sequentially through a series of angles obtaining readings at each angle. In this way, the electronic instrumentation 100 may obtain one and two dimensional measurements and then based on those measurements estimate the position and/or volume of the foreign body 10. It is also contemplated that the instrument can also be moved along the longitudinal axis of the instrument to take a series of measurements. For example, the distal portion 106 may be moved from a first sensing point at surface 12 of the tissue 11 to a second sensing point spaced from the surface 12. The sensing point spaced from the surface may be within the tissue 11 or outside of the tissue.

In at least one embodiment the electronic instrumentation 100 is adapted for rotation about its longitudinal axis to obtain readings at a plurality of angles. The more readings that are obtained at the different angles, the more accurate the data regarding the position and/or size of the foreign body 10 will be. For example, in one embodiment the electronic instrumentation 100 is rotated through 360 degrees about the longitudinal axis to obtain data. In another aspect, measurements are taken at a set number of angles. For example, where two measurements are taken it may be advantageous to obtain readings at a first angle and then at a second angle substantially perpendicular to the first angle. For another example, where three measurements are taken, readings may be obtained at a first angle, then at a second angle approximately 45 degrees offset from the first angle, and then at a third angle approximately 45 degrees offset from the third angle. Moreover, one or more rotations may be conducted at a first position on the surface, then the distal portion 106 moved about the surface 12 to a second position and another series of rotations may be conducted to assess the foreign body 10 within the tissue 11. These exemplary angles are for illustration purposes only. It is fully contemplated that in alternative embodiments electronic instrumentation may obtain data from any number of different angles and combinations thereof.

An accelerometer or gyroscope may be utilized to help determine the amount of rotation performed or indicate to the electronic instrumentation 100 when to stop taking readings. For example, the electronic instrumentation 100 may start obtaining readings and continuing obtaining readings as it is rotated about its longitudinal axis. Once the accelerometer or gyroscope detects that the electronic instrumentation 100 has made a full 360 degree rotation it may automatically stop the readings or emit a signal, such as an audible beep, to the operator to stop obtaining readings. Then based on the data obtained over the range of angles, the electronic instrumentation can provide an accurate assessment of the foreign body 10, including such things as three-dimensional size, shape, and location.

Further, in combination with, in addition to, or in lieu of an accelerometer or gyroscope the electronic instrumentation 100 may utilize a fiducial marker assembly. Fiducial markers can enhance the readings obtained by the electronic instrumentation 100 by providing precise location information for the foreign body 10 within the tissue 11. U.S. Pat. No. 6,235,038 issued to Hunter et al. and assigned to Medtronic Surgical Navigation Technologies includes disclosure regarding the use of fiducial markers and is incorporated herein by reference in its entirety. The fiducials may be of any appropriate type including optical reflectors, electrical coils, transmitters, electromagnetic, etc. Further, their placement with respect to the sensing end or distal portion 106 of the electronic instrumentation may be modified to suit the particular application. In this regard, the fiducial markers can even provide sufficient data to create 3-D images of the tissue 11 and the foreign body 10. This may be especially advantageous in the case where treatment requires removal of the foreign body 10. For example, the fiducial markers can allow creation of a 3-D image or model of the tissue 11, including the foreign body 10, from which the foreign body may then be evacuated. A second reading may be taken using the electronic instrumentation 100 and fiducial markers after attempting to remove the foreign body 10. Based on the second reading, the physician may determine the relative success of the removal (e.g., whether all of the pieces of the foreign body were properly removed). In this manner, using the fiducial markers can allow the physician to not only determine if any pieces of the foreign body 10 remain, but also know precisely where any unwanted foreign body pieces are located. The doctor can note those areas that contain fragments of the foreign body 10 that still need to be removed and then attempt to remove them. This process can be repeated until the foreign body 10 is removed to the surgeon's satisfaction. This allows for successful removal the entire foreign body 10.

The data obtained by the instrument 100 may be transmitted to an image guided surgery (IGS) system such that the data sensed by the instrument concerning the location and size of the foreign body may be integrated with the positioning data of the IGS system. Thus, a composite three-dimensional image showing the tissue and the boundaries of the foreign body can be calculated. The image may be displayed separately or as part of a composite image with the IGS display. The data from instrument 100 may be transmitted wirelessly or by wired communication to the IGS system. Alternatively, the instrument 100 may include a memory for recording the sensed data, from which the data may later be transferred to the IGS system or other device. A port, such as a USB port, may be provided to connect the instrument to the IGS system or other computer system to download the sensed data. In a further embodiment, the instrument 100 is a component of an IGS system. In this embodiment, sensor 100 is utilized to map the three-dimensional boundaries of the foreign body and the three-dimensional location of the foreign body relative to the patient's tissue. The IGS system then guides the user to remove all or substantially all of the foreign body based on the sensed data. In an alternative system, the IGS system includes an automated removal device in communication with the IGS system. The automated removal device is advanced to the foreign body site under computer control, activated to remove the foreign body under computer control and removed from the tissue.

In a further aspect, the IGS system automatically locates a void created by removing the foreign body and fills the void with an appropriate filler material. For example, where a foreign body is removed from a bone, the IGS system may fill the resulting void with a bone-growth promoting substance. Further, in at least one aspect a sensor may be placed in the filler material to verify optimal filling of the void. In other embodiments, the electronic instrumentation may be utilized to monitor the removal of the foreign body and subsequent filling of the resulting void. In an additional aspect, electronic instrumentation is used to detect proper packing of the void with bone filler material disposed between the bone filler material and the boney boundary. The instrumentation may detect spaces and/or foreign materials preventing the appropriate filling of the void. In still a further embodiment, the sensing instrument 100 is provided in combination with a tamp. In use, this embodiment allows the surgeon evaluate the backing of material in the void and apply pressure with the tamp to force filling material into any sensed spaces.

In addition to use with an IGS system or as stand alone components, in alternative embodiments the sensing instrument 100 includes one or more neuro integrity monitoring (“NIM”) electrodes disposed adjacent distal end portion 106. A neuro integrity monitoring system, with one or more sensors positioned on or in the patient may then be used to detect the presences of nerves near the NIM electrodes. The presence of nerves near distal end portion 106 may be communicated to the user through display on an IGS system, and/or through tactile, audible, or visual indicators used alone or in combination. In still a further aspect, instrument 100 includes a Doppler enabled sensing array to detect the blood flow or pulse within the patient. This Doppler information is provided to the user to indicate the proximity or direction of blood vessels. In addition, the absence of blood flow through blood vessels can indicate to the user that the blood vessel is constricted or severed. This information may be particularly useful as the user evaluates whether to dislodge a foreign body and whether when doing so may result in significant bleeding because the foreign body is acting as a tamponade in stopping blood flow.

In still a further aspect of the present invention, the sensing instrument is utilized to detect the track or path of a penetrating foreign body. In one aspect, the sensor is placed in the opening in the skin associated with a penetrating injury. As the sensor is advanced, it detects the path of the penetrating object by interrogating the surrounding tissue and detecting anomalies in the nature tissue associated with the passage of the foreign body. In this manner, the sensor instrument 100 is used to trace the path through the body to the foreign body. Thus, the foreign body may be removed through the same path as it entered the body thereby eliminating the need to create a secondary injury through previously unaffected tissue to remove the foreign body. Further, treating compounds such as antibiotics, coagulants, etc., can be inserted through the projectile path to treat the path and/or the tissue adjacent the foreign body. Further, when the foreign body is not removed, a polymer or other material is injected to partially or completely encapsulate the foreign body. Alternatively, an ablation device may follow the path or track detected by sensing instrument 100 either by IGS guidance or along a guide wire. The ablation device can then be energized to ablate the foreign body. Still further, in another aspect, the sensing instrument 100, either alone or in combination with an IGS system, may be used to detect the track or path of the penetrating foreign body without extending into or along the path. In one aspect, interrogation of the suspected path occurs by passing the energy through the skin without penetrating the skin. In this manner, the track or path of the foreign body can be determined and the user can evaluate what internal structures may have been impacted during the penetration and whether it is reasonable to remove the foreign body through the existing entry path or to create an alternative path that is less traumatic to the patient.

In a further aspect, the information gathered by the sensing instrument 100 is displayed on a display device external to the sensing instrument. In one aspect, location and detection information from multiple sensing and/or imaging systems as described above are simultaneously displayed on the external display device. In a further aspect, the external display device is a heads-up display worn by the user.

In still a further embodiment, the sensing instrument 100 provides localization and detection data in conjunction with one or more other sensors positioned in or on the body. Examples of such sensors are set forth in U.S. patent application Ser. No. 11/356,687, filed Feb. 17, 2006, entitled Sensor and Method for Spinal Monitoring, incorporated herein by reference in its entirety. In this embodiment, the additional sensors may be used to listen to the acoustic energy generated by sensing instrument 100 and detect changes in the acoustic patterns caused foreign bodies.

In other embodiments, the acoustic beam produced by the transducer 112 may be of any shape to facilitate obtaining data from the tissue, including but not limited to substantially conical or cylindrical shapes. As shown in FIG. 5, the beam may be substantially cone shaped. Use of a cone shaped beam is advantageous when a minimal number of readings is wanted as more data can be obtained from a cone shaped beam as compared to the fan shaped beam previously described. As shown in FIG. 6, in one embodiment the acoustic beam is a focused beam of substantially cylindrical shape. Further, it is contemplated that a single transducer or multiple transducers within the electronic instrumentation may be capable of producing various types of beams depending on the type of tissue being examined and/or the type of foreign body being detected. The treating physician or technician may have the ability to choose the appropriate beam on a case-by-case basis. Although not shown in FIGS. 5 and 6, in another embodiment the beam is directed substantially perpendicular to the longitudinal axis of the instrument such that is senses to the side of the instrument.

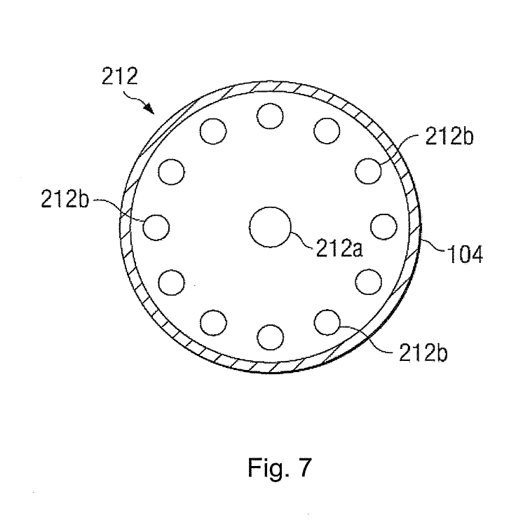

As shown in FIG. 7, it is also contemplated that in yet another embodiment that the electronic instrumentation 100 may include an array of transducers 212 located adjacent the distal portion 106 and disposed radially around the longitudinal axis. Where the array of transducers 212 is present there may be a dedicated receiving transducer 212a for detecting the echo from the array of emitting transducers 212b. Each of the emitting transducers 212b may emit an acoustic signal at a different frequency to allow the receiving transducer to distinguish between return signals. In an alternative design, the array 212 is phased or timed such that the receiving transducer 212a is detecting a single echo at a time correlated to a single emitting transducer 212b. To this end, it is fully contemplated that the electronic instrumentation 100 includes a timing means for synchronizing the emitting and receiving of acoustic signals.

The electronic instrumentation 100 also includes an output 118. In some embodiments, the output 118 is visual display, such as a liquid crystal display, LED, or other visual display. The display may provide such information as the presence of a foreign body, the estimated depth of a foreign body, the estimated size of a foreign body, and/or the estimated position of a foreign body. In lieu of or in addition to a display, the electronic instrumentation 100 may include other types of outputs 118. In general, the output 118 is capable of outputting data in a human intelligible form and/or outputting data to a separate device that produce the data in a human intelligible form. For example, the data is transmitted to an external display device, such as a head mounted display. Still further, the instrumentation may include an audible output, such as a speaker. In one embodiment, the audible output beeps or makes other sounds indicating the presence of a foreign body. The intensity, volume, pitch, length, or other characteristic of the sound may indicate the relative depth of the foreign body and/or the relative size of the foreign body. Other human intelligible forms, such as vibrations, are also contemplated as means of outputting tissue data. In another embodiment, the output may be a wireless communication mechanism. In this regard, the electronic instrumentation 100 may be configured to transfer data using RFID, inductive telemetry, acoustic energy, near infrared energy, “Bluetooth,” computer networks, other wireless communication mechanisms, and combinations thereof. The electronic instrumentation 100 may transfer data wirelessly to offload tasks such as the computing performed by the signal processor, displaying the data, and/or storing the data. Alternatively, the instrument may configured for transferring data over a wired connection or output.

The electronic instrumentation 100 includes a power supply 116. In one embodiment, the power supply 116 may be an internal power source. That is, the power supply 116 may be fully disposed within the electronic instrumentation 100. The internal power source may be a battery or a plurality of batteries. In an alternative embodiment, it is also fully contemplated that the electronic instrumentation 100 may be adapted to receive power from an external source. For example, it is fully contemplated that the electronic instrumentation 100 receives power from a wall socket or other common power source through a wired connection. To this end, the electronic instrumentation 100 may itself include a wire adapted to plug into the power source. On the other hand, the electronic instrumentation 100 may include an adapter or receiver for selectively connecting to a wired power supply, such that the instrumentation is not permanently attached to the wire. In these embodiments, it is contemplated that the electronic instrumentation 100 receives power via a Universal Serial Bus (“USB”) system. In this way the electronic instrumentation 100 may be adapted to communicate over a USB cable with an external device, such as a laptop or desktop computer, to receive power and also transmit data. In such embodiments, the electronic instrumentation 100 may utilize the computing power of the external device to perform the signal processing and output functions. In this regard, it is contemplated that the external device may also be a handheld device such as a cell phone, PDA, BlackBerry, or similar type device. It is fully contemplated that the electronic instrumentation 100 may be configured to include as few parts as needed, utilizing the features of the external device to the full extent possible. This can be very beneficial where the electronic instrumentation 100 is adapted to be disposable such that cost is kept to a minimum.

In still a further embodiment, it is contemplated that the electronic instrumentation 100 is adapted for placement within or in combination with a tissue removal instrument or other medical device. For example, in some embodiments the electronic instrumentation 100 is adapted for use with a guide wire. In one embodiment, the housing of the electronic instrumentation 100 includes an opening for receiving a guide wire. In particular, the opening may be oriented such that when the transducer of the electronic instrumentation is in substantial alignment with the foreign body, the guide wire may be passed through the opening directly towards the foreign body. In such instances, a tool for removing the foreign body can then be guided precisely to the foreign body by the guide wire. In a further aspect, a protective sheath is provided to surround sensing instrument 100. The sheath surrounds at least a portion of the sensing instrument while it is advanced to the foreign body and remains in place after removal of the sensing instrument. The protective sheath then provides a passage guiding instruments to the foreign body or surgical site through a protected channel. The protective sheath is a fixed diameter cannula, a flexible membrane, an expandable tissue dilator that can be enlarged to form a passage larger than the diameter of the sensing instrument, or other tubular members.

As another example, placement of the electronic instrumentation 100 within or in combination with an instrument, such as a curette, brushes, burrs or laser tissue ablation device, may be particularly advantageous where the instrument is used to remove the foreign body and the electronic instrumentation 100 is utilized to determine the location of the foreign body. To the extent that the electronic instrumentation is used in combination with another medical device, it is contemplated that the electronics are incorporated into a sheath, film, or other type of casing designed to engage the medical device without impairing the function of the medical device. In still a further embodiment, instrument 100 is incorporated with or into a minimally invasive surgical system. For example, in this embodiment the sensing features of the present system are added to powered abrader and cutters such as the Visao® High Speed Otologic Drill and XPS®, Magnum®, Straightshot®, Microdebriders offered by Medtronic Xomed, Inc. The transducer of the instrumentation 100 would be positioned adjacent the cutting end of the cutter and in one aspect, extend beyond the cutter. The tissue removal device may utilize ultrasound to ablate tissue as disclosed in U.S. Pat. No. 6,692,450 to Coleman incorporated by reference herein in its entirety.

In another aspect the electronic instrumentation 100 is utilized with blind cutting instruments having their cutting elements disposed out of the line of sight from the user. For example, the transducer(s) may be placed on the angled portion of the cutting instruments disclosed in U.S. Pat. No. 6,544,749 to Mitusina, et al, incorporated by reference herein in its entirety. In still another embodiment, the transducer(s) of the instrumentation 100 may be combined with a lens or camera (not shown) for visualization of tissue and/or foreign body adjacent the working end of the foreign body removal device. In this embodiment, the electronic instrumentation 100 provides feedback concurrently with the video image displayed by the camera to offer the surgeon additional information on tissue type and location of the foreign object. For example, the electronic instrumentation 100 may be used with a rigid endoscope, a flexible endoscope, a fiberoptic visualization device, and/or other video devices. In yet a further embodiment, the instrument provides a proprioceptive (tactile) response to the user based on the sensed data to indicate to the user in an intuitive manner the type of tissue or foreign body being encountered proximal the tissue removal device. In a further form, the instrument 100 includes one or more forward looking sensors or transducers that alert the user through proprioceptive response of nearing collisions with other implants or vital tissues, such as nerves and blood vessels in the vicinity of the tissue removal device. For example, but without limitation, the proprioceptive signals may include vibrations, lights, and sounds, alone or in combination. Further, each of these signals may be controlled to become more intense as the distance between the removal device and vital tissue decreases indicating an imminent danger of collision. Further, when combined with an IGS system, the sensed data may be incorporated into an image display to assist the surgeon in guide the instrument to avoid vital tissues.

In a further aspect, the sensing instrument is used to locate foreign bodies intentionally placed in the body. For example, when removing an implanted bone growth stimulator or nerve stimulator, the sensing instrument is useful to locate lead wires leading to electrodes implanted in the bone or on nerves. The leads can then be served immediately adjacent the electrode without disturbing the bone or nerve. Still further, the sensing instrument can be used to locate portions of an implant system needed to be removed or adjusted. For example, in a spine stabilization procedure, a fixation system is implanted. At a later date the system needs to be adjusted to provide more or less stabilization. The sensing instrument is used to locate the screw or other fixation element to be removed or adjusted to permit a static fixation system to operate as a more dynamic system. In still a further aspect, the sensing instrument can be used to detect foreign bodies associated with an implantation procedure. For example, during a vertebroplasty, bone cement is injected into the vertebral body. In some patients, bone cement may unintentionally pass into the spinal canal. The sensing instrument is used to detect the presence of bone cement in the spinal canal and may further be used to detect the size or volume of material present and/or the extent of impingement on the nervous system. With this information, the care provider can undertake remedial measures if necessary.

It is fully contemplated that the electronic instrumentation 100, whether used as a stand-alone unit or in combination with another medical device, may be disposable. That is, the electronic instrumentation 100 is designed for use in only one medical procedure or for a limited amount of time. For example, in one aspect the electronic instrumentation 100 includes a circuit that breaks or disconnects if the instrumentation is subjected to autoclaving or other types of sterilization procedures. The electronic instrumentation 100 may also include a battery with a predetermined life. For example, the battery may be designed to provide power to operate the electronic instrumentation for 8 hours after initiation. This would give the electronic instrumentation sufficient power for long surgical procedures, yet limit the useful life of the instrumentation to single use applications. The length of the battery life may be more or less than 8 hours in other embodiments.

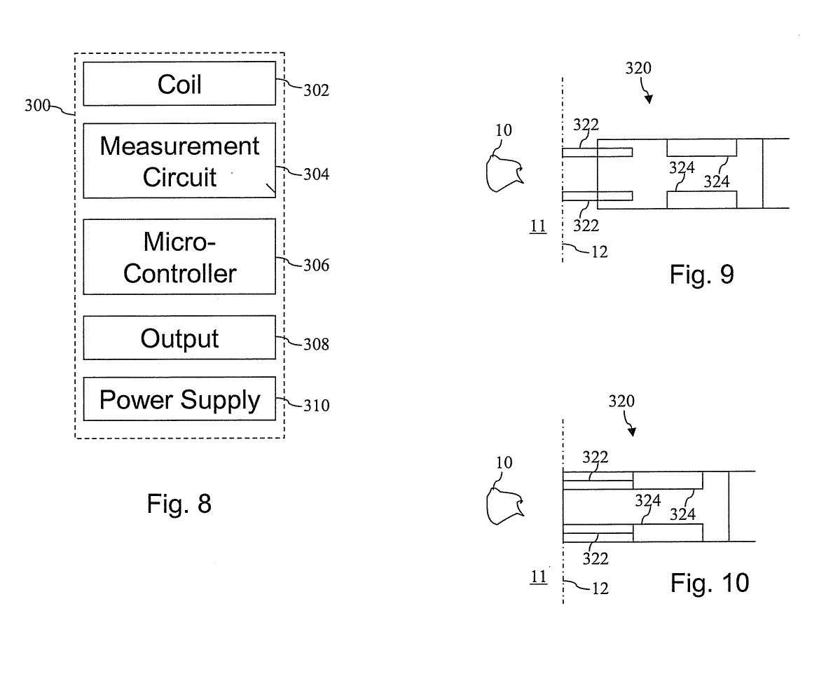

Referring now to FIG. 8, shown therein is a schematic view of an instrument 300 according to one embodiment of the present disclosure. In general, the instrument 300 is configured to detect the presence of the foreign object 10 within the tissue 11. However, instead of monitoring reflected energy signals, the instrument 300 determines the presence, location, and/or size of the foreign object 10 based on changes to a characteristic of a circuit or coil of the instrument 300. In that regard, as shown the instrument 300 includes a coil 302, a coil measurement circuit 304, a micro-controller or processor 306, an output 308, and a power supply 310. In this embodiment, the inductance, eddy current, circuit “Q,” or other characteristic of the coil 302 and/or coil measurement circuit 304 may be monitored for changes. Changes in the characteristics of the coil 302 and/or circuit 304 may be indicative of the presence of a ferrous metal in proximity to the instrument 300. In other embodiments, the instrument 300 may be used to detect other materials, including ferrous and non-ferrous metals. In particular, the micro-controller 306 may continuously monitor and compare the values of the characteristic(s) of the coil 302 and/or circuit 304 as the instrument 300 is passed over the surface 12 of the tissue 11. Based on the changes in the characteristics, the micro-controller 306 may determine when the coil 302 is in closest proximity to the foreign body 10 and/or when the instrument 300 is substantially aligned with the foreign body. In this manner, the instrument 300 may be utilized to detect the foreign body 10 within the tissue 11. In at least one embodiment, the instrument 300 functions by monitoring the voltage through the coil 302 compared to the voltage being supplied to the coil 302 by the circuit 304. In at least one embodiment, the instrument 300 functions by monitoring the differential in the voltage through the coil 302 compared to the voltage through a fixed value coil or resistor of the circuit 304.

The size, shape, windings, core, and/or other features of the coil 302 may be configured for the particular type of foreign body to be detected. In that regard, generally the larger the size of the foreign body to be detected, the larger the size of the coil. On the other hand, the smaller the size of the coil, generally the more sensitive the coil will be and, therefore, smaller coils may provide higher resolution data regarding the position and size of the foreign object. For this reason, the coil 302 may be modular component such that a coil with the appropriate features may be utilized depending on the circumstances. In that regard, it is contemplated that the surgical instrument 300 may include a plurality of interchangeable coils with varying features. Further, in some embodiments a user may begin with a larger coil to detect generally the location and presence of the foreign body and then move to a smaller coil to obtain higher resolution information about the location and/or size of the foreign body.

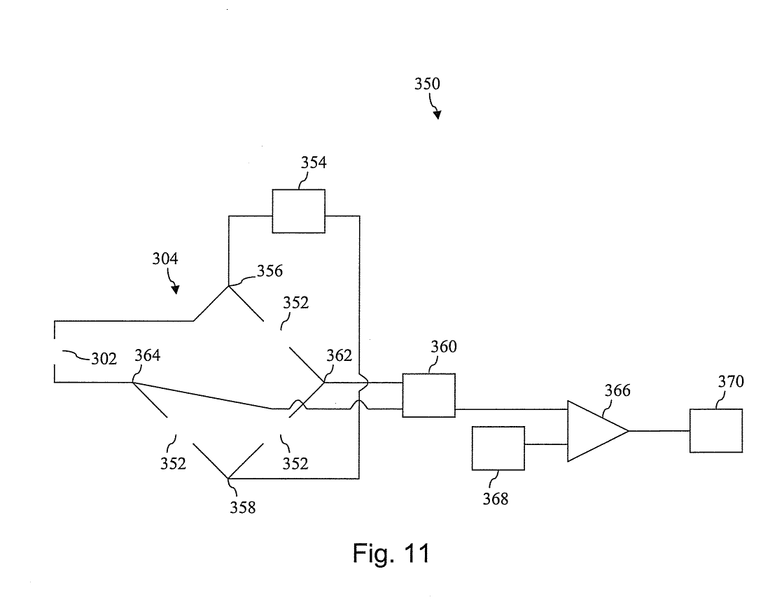

In response to the presence of the foreign body 10 and/or alignment of the instrument 300 with the foreign body, the output 308 may produce an alert or otherwise output data indicating the presence and/or location of the foreign body. The output 308 may be similar to the output 118 described above and, therefore, will not be described in detail here. 063 The instrument 300 may also include accelerometers, gyroscopes, fiducial markers, and/or other mechanisms for obtaining 2-D and/or 3-D positioning data for the foreign object. In one particular embodiment, the instrument 300 is moveable between at least two positions relative to the surface of the tissue and includes a mechanism for indicating what position the instrument is in. For example, referring to FIGS. 9 and 10, shown therein is an example of a spring-loaded tip system 320 that can provide a signal indicative of what position the instrument 300 is in. In the current embodiment, the instrument 300 is moveable between two positions in contact with the surface of the tissue. In the first position and as shown in FIG. 9, only probe tips 322 touch the surface. In the second position and as shown in FIG. 10, a substantial portion of the active end of the instrument 300 is in contact with the surface. When second position, the probe tips 322 are retracted and contact a pair of switch contacts 324. When the switch contacts 324 are triggered by the probe tips 322, the micro-controller 306 is notified that the instrument is in the second position and can then categorize the data accordingly. A user can then take measurements at each of the positions and compare the values of the characteristic(s) of the coil 302 and/or circuit 304. Based on the differences in the values of the characteristic(s) of the coil 302 and/or circuit 304 between the two positions, the depth of the foreign body may be more accurately determined than with a single measurement. In other embodiments, the instrument 300 may include additional positions for even more accurate determination of the depth of the foreign body. Further, the probe tips 322 and switch contacts 324 are merely one example of a way to monitor the relative positions of the instrument 300. Numerous other mechanisms may be employed, such as but not limited to using hall effect transducers, infrared systems, accelerometers, laser systems, and other systems for providing positioning data.

A similar multi-position approach may be employed with the electronic instrumentation 100 described above. In one embodiment, the electronic instrumentation 100 includes a compressible coupling medium between the transducer and the tissue. The compressible coupling medium serves to retain the coupling between the transducer and the tissue even when the transducer is not in direct contact with the tissue surface. The position of the transducer relative to the tissue surface may be determined by the compression state of the compressible coupling medium. In other embodiments, the position of the transducer relative to the tissue surface may be determined by an accelerometer or gyroscope that is adapted to monitor initial and successive positions.

In a further aspect, the sensing instrument uses the difference in heat to detect and/or localize foreign bodies. In this embodiment, the sensing instrument includes an RF energy electrode to transmit RF energy to surrounding tissues. The RF energy is absorbed by the tissues and is also absorbed or reflected by any foreign bodies at a different rate. The resulting difference in temperature is detected by the sensing instrument allowing a determination to be made concerning the presence and/or location of the foreign body.

Referring now to FIG. 11, shown therein is a schematic illustration of a circuit 350 for use in detecting a foreign body according to one embodiment of the present disclosure. In the current embodiment, the circuit 350 functions as an analog circuit. However, in other embodiments the concepts of the circuit 350 may implemented in a digital circuit. As shown, the circuit 350 includes coil 302 and an exemplary embodiment of circuit 304. It should be noted that circuit 304 is merely one example of the type of circuit that may be used with the present disclosure and should in no way be considered limiting. Numerous other suitable circuit designs would be apparent to one skilled in the art based on the present disclosure. As, shown the coil 302 is a component of the circuit 304. In particular, the circuit 304 includes the coil 302 and a plurality of fixed value coils or resistors 352. A voltage source 354 is connected to the circuit 304 at nodes 356 and 358. In some embodiments, the voltage source 354 is configured to provide a voltage to the circuit having a frequency between 1 Hz and 20 MHz. In other embodiments, the voltage source may provide a voltage having a frequency outside of that range. The precise frequency chosen may be based on the coil 302, the type of foreign body to be detected, the type of tissue being interrogated, and other factors. In some embodiments, the frequency may be manually selected by the user. In some embodiments, the voltage may have a varying frequency. In some embodiments, a varying frequency may facilitate a more accurate identification of the foreign body and/or its location. In yet other embodiments, the frequency of the voltage source is not critical. In such embodiments, the actual value of the voltage may be utilized to monitor the presence of the foreign object.

A voltage differential amplifier 360 is connected to the circuit 304 at nodes 362 and 364. In other embodiments, the circuit 350 does not include the voltage differential amplifier 360. The voltage differential amplifier 360 amplifies the voltage differential caused by the presence of a ferrous foreign body near the coil 302. In that regard, the circuit 304 including the coil 302 an electromagnetic field into the tissue. The interaction between the electromagnetic field and the ferrous foreign body causes the inductance or other characteristic of the coil 302 to vary. This variance results in a detectable voltage differential. However, in many instances the voltage differential may be very slight, on the order of mV or μV in some instances. Thus, in some embodiments the voltage differential amplifier 360 amplifies the voltage differential before passing it on to a comparator 366. The comparator 366 in turn compares the voltage differential with a predetermined threshold. The predetermined threshold is defined by a sensitivity control 368. The predetermined threshold of the sensitivity control 368 may be based on the size, type, and/or depth of a foreign body to be detected. In some embodiments, the sensitivity control 368 and its corresponding threshold may be set by the user. In the event that the voltage differential is above (or below in some embodiments) the predetermined threshold, the comparator 366 will send a signal to an output 370. The output 370 can alert the user to the presence of the foreign body and, in some embodiments, may further provide the approximate depth, size, or other characteristic for the foreign body.

In some embodiments, the instrument 300 may include a plurality of coils 302 and circuits 304. The plurality of coils 302 and circuits 304 may function as an array and/or operate as redundancies. In such embodiments, the coils 302 and circuits 304 may be multiplexed. Further, in such embodiments, each of the coil/circuit combinations may have its own output. In that regard, depending on the arrangement of the coils the order in which the output mechanisms provide alerts to the user can provide additional data regarding the location of the foreign object. For example, in some embodiments each sensing coil of the array may have a dedicated LED output. Further, the LED outputs may be arranged in a substantially similar manner to the coil array. Thus, as the instrument 300 approaches the foreign object, first the nearest coil will produce visual alert through the LED. As the instrument continues to approach the foreign object, the other coils will subsequently produce an alert. The user can then monitor the location of the foreign object moving the instrument 300 around the area adjacent the foreign object and following the active LEDs of the coil array.

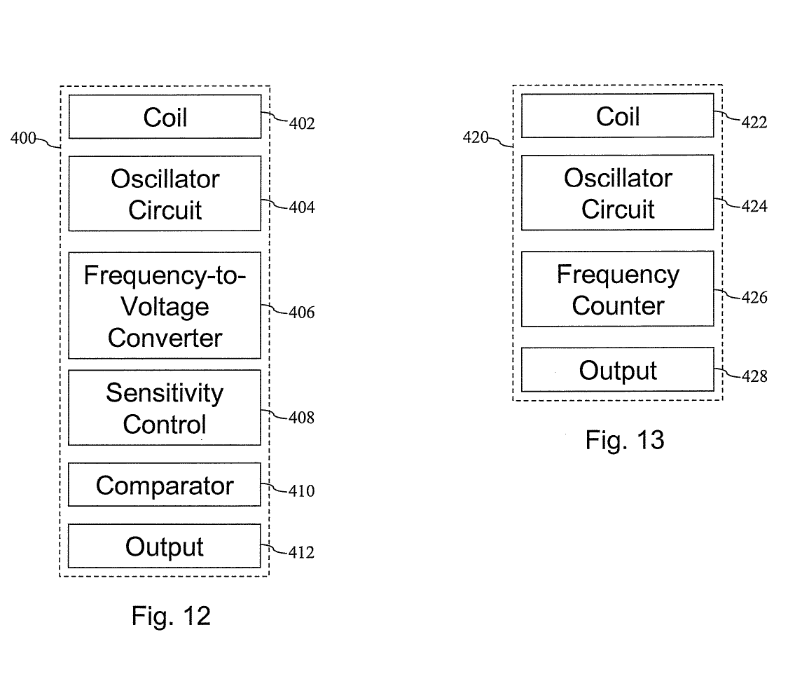

Referring now to FIG. 12, shown therein is a schematic illustration of an instrument 400 according to one embodiment of the present disclosure. In some aspects, the instrument 400 may be similar to the other instruments described in the present disclosure. Thus, some aspects of the instrument 400 will not be described in great detail. The instrument 400 includes a coil 402, an oscillating circuit 404, a frequency-to-voltage converter 406, a sensitivity control 408, a comparator 410, and an output 412. The coil 402 may be part of the oscillating circuit 404. In general, the oscillating circuit 404 provides a varying voltage across the circuit and in particular through the coil 402. In some embodiments, the oscillating circuit has a substantially sinusoidal pattern. However, in other embodiments it may have other patterns. The frequency-to-voltage converter 406 monitors the frequency of the voltage passing the through the coil 402. In particular, the frequency-to-voltage converter 406 converts the frequency of the voltage passing through the coil 402 into a corresponding voltage. The frequency of the coil 402 may vary from that of the oscillating circuit to the presence of a ferrous foreign object. The corresponding voltage provided by the frequency-to-voltage converter 406 may then be passed to the comparator 410. If the corresponding voltage is above the threshold set by the sensitivity control, then the comparator will signal to the output 412 to produce an alert and/or provide data related to the detection of the foreign object. If the corresponding voltage is not above the threshold, then no alert will be produced.

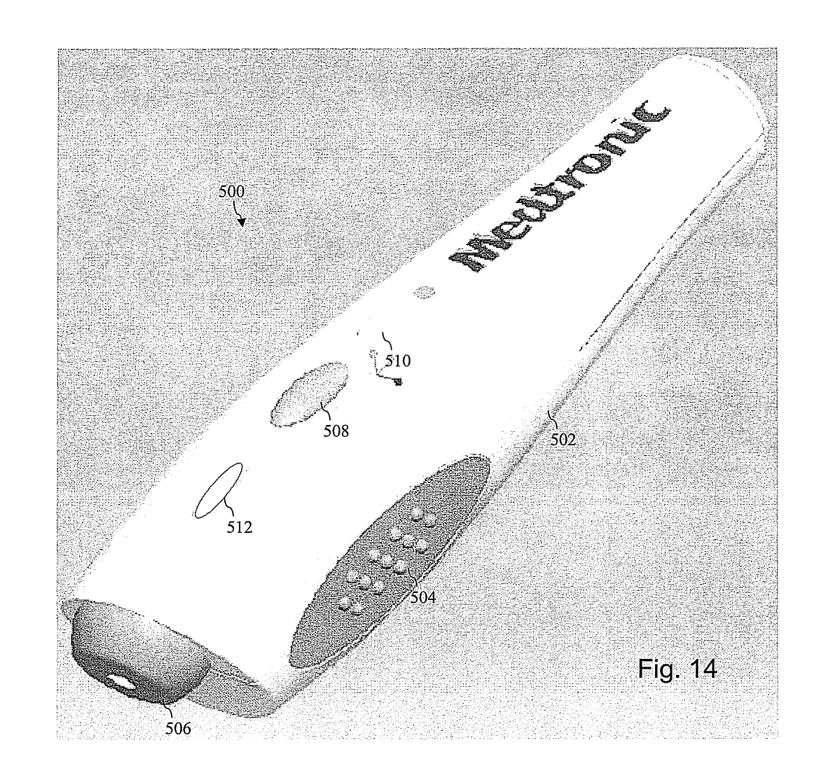

Referring now to FIG. 13, shown therein is a schematic illustration of an instrument 420 according to one embodiment of the present disclosure. In some aspects, the instrument 420 may be similar to the other instruments described in the present disclosure. Thus, some aspects of the instrument 420 will not be described in great detail. The instrument 420 includes a coil 422, an oscillating circuit 424, a frequency counter 426, and an output 428. The coil 422 and the oscillating circuit 424 function substantially similar to the coil 402 and the oscillating circuit 404 described above. The frequency counter 426 functions by monitoring the number of counts at a given frequency over a time period. Based on the changes in the frequency and the number of counts at a given frequency, the frequency counter can determine the coil's 422 proximity to the foreign object.

Whereas the measurements of the instrument 400 were substantially absolute (i.e., either above the predetermined threshold or not), the measurements of the instrument 420 are more relativistic (i.e., compares counts at different frequencies). In some embodiments, the features of the instrument 400 and the instrument 420 may be combined in a single instrument. In such an embodiment, the absolute measurements of the instrument 400 can provide a general indication that a foreign object is present and then the relativistic measurements of the instrument 420 can provide more precise data regarding the actual location of the foreign body within the tissue.

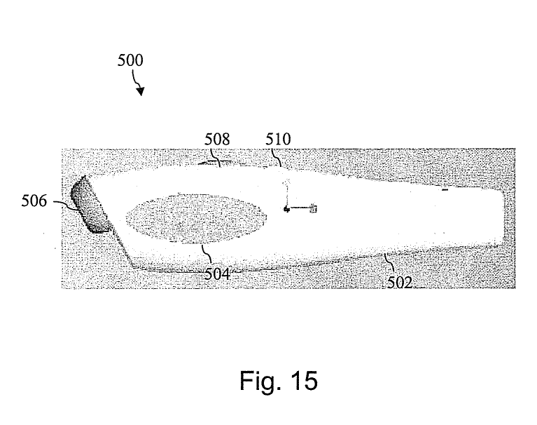

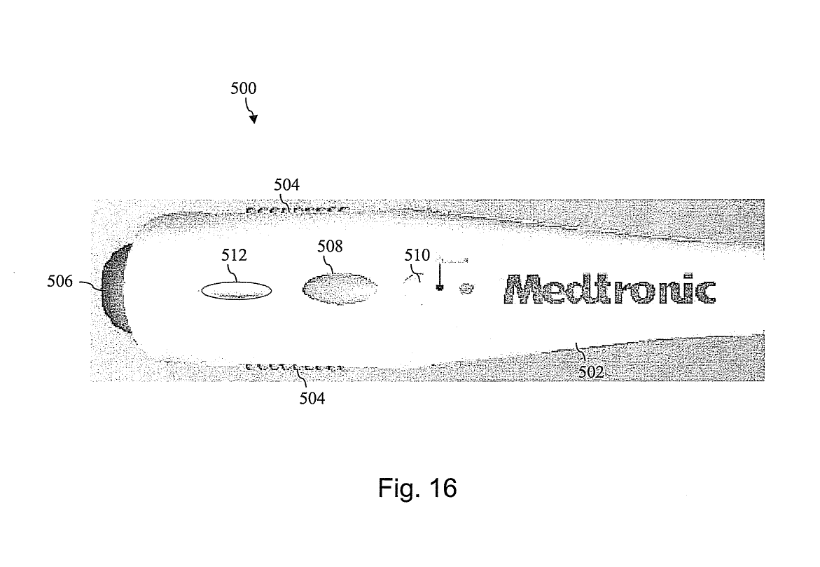

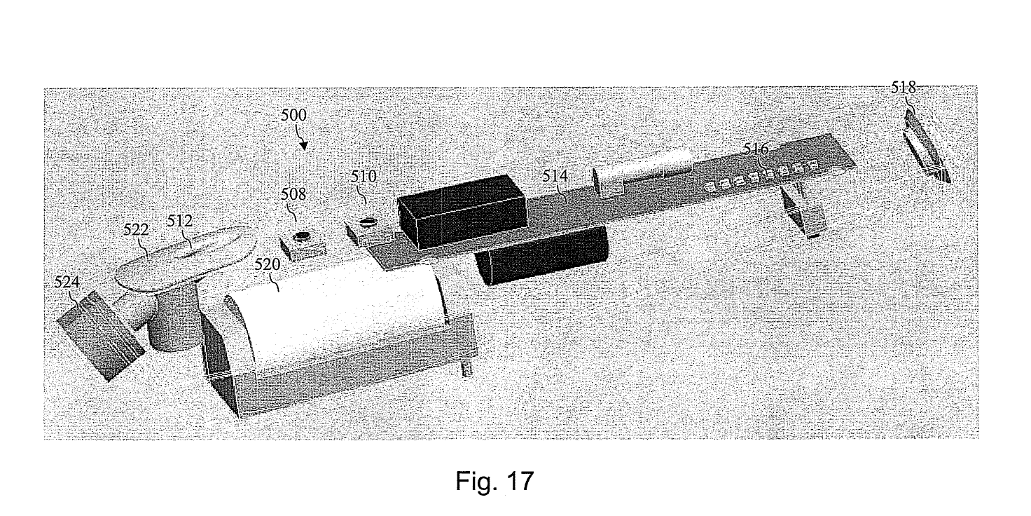

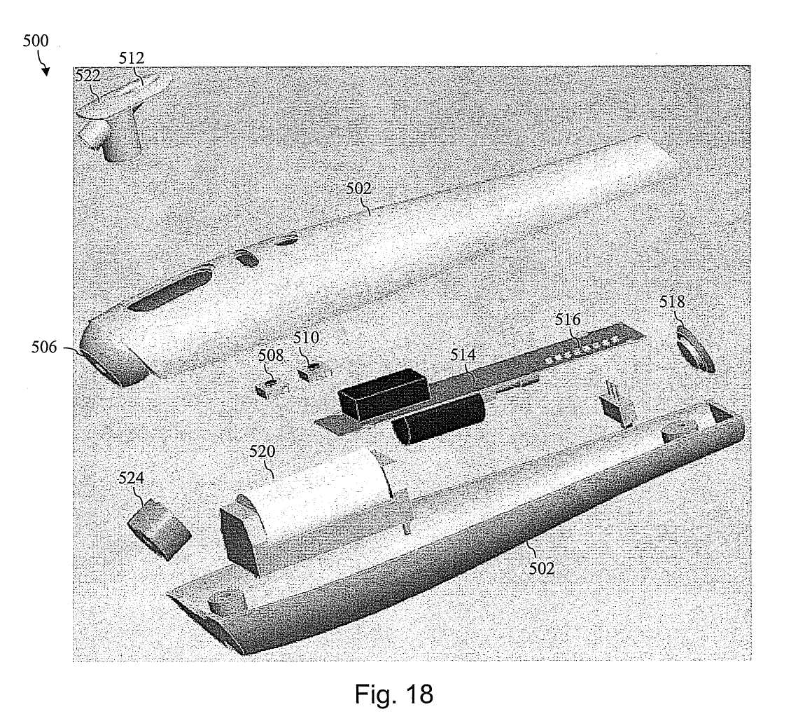

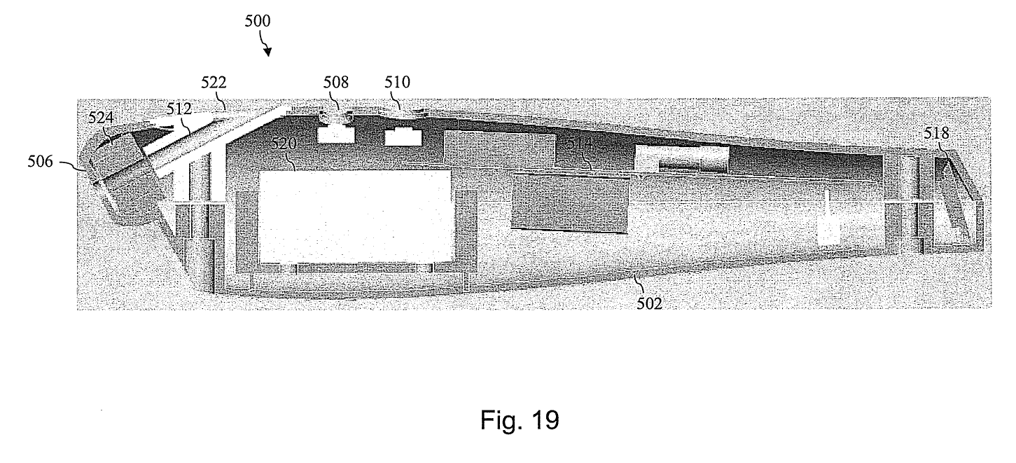

Referring now to FIGS. 14-19, shown therein is one embodiment of an instrument 500 incorporating aspects of the present disclosure. FIG. 14 is a perspective view of the instrument 500; FIG. 15 is a lateral view of the instrument; FIG. 16 is a top view of the instrument; FIG. 17 is a perspective cutaway view of the instrument; FIG. 18 is an exploded view of the instrument; and FIG. 19 is a cross-sectional side view of the instrument.

Referring more specifically to FIGS. 14-16, the instrument 500 includes a housing 502 having gripping portions 504. The gripping portions 504 include roughened surfaces to facilitate grasping by a hand of a user. The instrument 500 also includes a sensing portion 506. In general the sensing portion 506 is adapted to house or maintain the transducer(s) and/or coil(s) of the instrument. The instrument 500 also includes buttons 508 and 510. The buttons may be utilized for many functions. For example, in some embodiments the buttons may serve as an on button, an on/off button, a null button, a reset button, a trigger button, and numerous other buttons and functions. Further, the instrument 500 may include more or less buttons in other embodiments. The instrument 500 also includes a passage 512. In some embodiments, the passage 512 is adapted to guide a guide wire (not shown) towards a detected foreign object. In such embodiments, after the guide wire has been appropriately positioned the instrument 500 may be retracted over the guide wire and another surgical instrument passed along the guide wire. In some embodiments, a surgical instrument for removing the foreign body maybe guided to the foreign body by the guide wire.

Referring more specifically to FIGS. 17-19, some of the inner components of the instrument 500 may be identified. The instrument 500 includes a circuit board 514 that serves as an interface between many of the components of the instrument 500. In the current embodiment, the circuit board 514 includes a plurality of LEDs 516. As described above, the LEDs 516 may serve as an output mechanism for the instrument 500. In addition to the LEDs 516, the instrument 500 also includes a speaker 518. In some embodiments, the instrument 500 also includes a tactile feedback system. The tactile feedback system may be incorporated into or connected to the circuit board 514 in some embodiments. The instrument 500 also includes an internal power supply or battery 520. A guide wire piece 522 defines the passage 512 described above. The guide wire piece 522 may be adapted to mate with transducer or coil element 524. In that regard, a portion of the guide wire piece 523 may pass into an opening of the element 524. The element 524 is sized to fit within the sensing portion 506 of the housing 502, as shown.

Though the electronic instrumentation and instruments have been described primarily in connection with detecting the presence, location, and size of a foreign body and determining whether removal of the foreign body was successful, the electronic instrumentation according the present disclosure may have many other applications. In one application, the instrument may be used after filling of a void with bone filling material to evaluate completeness of the filling. For example, the difference in material properties between the native bone, the bone filler and any substance left in the void may be sensed by the instrument. If a foreign substance, such as blood, air, saline solution, lesion, tumor, etc., remains after filling the instrument may detect it and alert the user.

In another application, the electronic instrumentation is configured to determine the actual density of tissue, rather than simply distinguishing between different types of tissue and/or foreign objects. This may be advantageous in the treatment of patients with osteoporosis. In this aspect, the electronic instrumentation is adapted to determine the size of other tissue features, such as lesions. Lesion in this sense is intended to include any type of abnormal tissue, malformation or wound related to a bone or other tissue, including cancers, voids, tumors, missile injuries, projectiles, puncture wounds, fractures, etc. For example, it is fully contemplated that the disclosed electronic instrumentation is useful to detect and determine the size of bone cancer voids, cancer cells, and tumors. In another aspect, the electronic instrumentation is used to probe suspect tissue and alert the user to the presence of anomalous tissue based on reflected energy indicating different densities. In still a further aspect, the electronic instrumentation is used to monitor the growth and healing of soft tissues, such as tendons and ligaments, as well as bone. Further, in another aspect the electronic instrumentation is utilized to create a 2-D or 3-D image of the tissue. Finally, the electronic instrumentation may be configured to perform a plurality of these applications in combination.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

1-30. (canceled)

31. A handheld surgical instrument for use in detecting a foreign body within a tissue, comprising:
a housing having a proximal portion, a distal portion, and a longitudinal axis extending therebetween;
a gripping portion positioned adjacent the proximal portion and adapted for grasping by a user;
a sensor circuit positioned within the distal portion, the sensor circuit configured such that a characteristic of the sensor circuit is modified by a proximity of the foreign body to the sensor circuit;
a processor for determining a position of the foreign body based on a value of the characteristic of the sensor circuit that is modified by the proximity of the foreign body to the sensor circuit.

32. The surgical instrument of claim 31, wherein the characteristic determined by the proximity of the foreign body to the sensor circuit is an inductance of the sensor circuit.

33. The surgical instrument of claim 32, wherein the proximity of a metallic foreign object to the sensor circuit determines the inductance of the sensor circuit.

34. The surgical instrument of claim 33, wherein the sensor circuit includes a bridge circuit having a plurality of coils and wherein the inductance of at least a portion of the bridge circuit is the inductance of the sensor circuit modified by the proximity of the metallic foreign body to the sensor circuit.

35. The surgical instrument of claim 34, further comprising an alternating current (“A/C”) source connected to the bridge circuit.

36. The surgical instrument of claim 35, wherein at least one of the coils has a variable inductance affected by the proximity of the metallic foreign object to the at least one coil.

37. The surgical instrument of claim 36, further comprising a comparator for determining whether the inductance of the bridge circuit is indicative of the presence of the metallic foreign body.

38. The surgical instrument of claim 37, further comprising an output device in communication with the comparator for producing an output in the event that the inductance of the bridge circuit is indicative of the presence of the metallic foreign body.

39. The surgical instrument of claim 38, further comprising a voltage differential amplifier in communication with the bridge circuit and the comparator.

40. The surgical instrument of claim 34, further comprising a plurality of bridge circuits positioned in an array, wherein the inductance of the plurality of bridge circuits of the array may be utilized to triangulate the position of the metallic foreign body.

41. The surgical instrument of claim 32, further comprising an accelerometer adapted to monitor the relative position of the surgical instrument with respect to an initial reference point.

42. The surgical instrument of claim 32, further comprising fiducial markers adapted to provide data for producing a three-dimensional image of the foreign body within the tissue.

43. The surgical instrument of claim 31, wherein the characteristic modified by the proximity of the foreign body to the sensor circuit is an eddy current of the sensor circuit.

44. The surgical instrument of claim 31, wherein the characteristic modified by the proximity of the foreign body to the sensor circuit is a circuit “Q” of the sensor circuit.

45. The surgical instrument of claim 31, further including an output mechanism adapted to produce an indicator of the presence of the foreign body.

46. The surgical instrument of claim 45, wherein the output mechanism is a visual display.

47. The surgical instrument of claim 45, wherein the indicator is an audible signal.

48. The surgical instrument of claim 31, wherein the processor is further adapted to determine a volume of the foreign body.

49. The surgical instrument of claim 31, wherein the processor is further adapted to determine a material characteristic of the foreign body.

50. The surgical instrument of claim 31, wherein at least one of the housing, sensor circuit, or processor is adapted to degrade during sterilization to limit the surgical instrument to single use applications.

51. The surgical instrument of claim 31, further comprising a mechanism for determining the distance between the sensor circuit of the surgical instrument and a surface of the tissue.

52. The surgical instrument of claim 51, wherein the mechanism is spring-loaded.

53. The surgical instrument of claim 52, wherein the mechanism is moveable between a first extended position at least partially outside of the housing and a second retracted position substantially within the housing.

54. A method of detecting a foreign body within a tissue, comprising:
providing a hand-held device having a sensor circuit and a processor, the sensor circuit configured such that a characteristic of the sensor circuit is affected by a proximity of the foreign body to the sensor circuit, the processor for determining a position of the foreign body based on the value of the characteristic of the sensor circuit affected by the proximity of the foreign body to the sensor circuit;
positioning the hand-held device adjacent the tissue;
monitoring the characteristic of the sensor circuit affected by the proximity of the foreign body to the sensor circuit;
producing an alert in response to a change in the characteristic of the sensor circuit indicative of the presence of the foreign body; and
determining the position of the detected foreign body within the tissue based on the value of the characteristic of the sensor circuit affected by the proximity of the foreign body to the sensor circuit.

55. The method of claim 54, further comprising moving the hand-held device about the surface of the tissue while monitoring the characteristic.

56. The method of claim 55, further comprising utilizing an accelerometer to determine the position of the handheld device relative to an initial point.

57. The method of claim 26, further comprising determining a volume of the foreign body.

58. The method of claim 57, further comprising:
utilizing the position and volume of the foreign body to guide a surgical instrument to the foreign body; and
removing the foreign body with the surgical instrument.

59. The method of claim 58, wherein the surgical instrument is electronically guided to the foreign body using the location and volume of the foreign body.

60. The method of claim 58, further comprising coupling the hand-held device to the surgical instrument prior to removing the foreign body with the surgical instrument.

61. The method of claim 58, further comprising inserting a guide wire through an opening in the hand-held device towards the foreign body.

62. The method of claim 61, further comprising guiding the surgical instrument along the guide wire to the foreign body.

63. The method of claim 62, further comprising removing the hand-held device from around the guide wire prior to guiding the surgical instrument along the guide wire.

64. A handheld surgical instrument for use in detecting a foreign body within a tissue, comprising:
a housing having an external gripping portion and a sensor portion having a conductive surface, the sensor portion adapted to be in conductive contact with a surface of the tissue;
an energy source adapted for emitting an energy signal into the tissue, the energy signal configured to pass through the tissue and at least partially reflect off a boundary between the foreign body and the tissue;
a sensor adapted for detecting the reflected signal; and
a processor for determining a characteristic of the foreign body based on the reflected signal;
wherein the characteristic of the foreign body determined by the signal processor includes a material attribute of the foreign body.

65. The surgical instrument of claim 64, wherein the material attribute of the foreign body is selected from the group consisting of ferrous and non-ferrous.

66. The surgical instrument of claim 64, wherein the material attribute of the foreign body is selected from the group consisting of metallic and non-metallic.

67. The method of claim 64, wherein the characteristic of the foreign body determined by the signal processor further includes the location of the foreign body within the tissue.

68. A handheld surgical instrument for use in detecting a foreign body within a tissue, comprising:
a sensing means for monitoring a proximity of the foreign body to the sensing means;
a processing means for determining a position of the foreign body within the tissue based on the proximity of the foreign body to the sensing means;
a housing means for containing the sensing means and the processing means; and
a gripping means for grasping by a user.

69. The surgical instrument of claim 68, wherein the sensing means is configured for sending and receiving energy signals.

70. The surgical instrument of claim 69, wherein the sensing means is configured for sending and receiving acoustic signals.

71. The surgical instrument of claim 68, wherein the sensing means is configured such that a characteristic of the sensing means is modified by the proximity of the foreign body to the sensing means.

72. The surgical instrument of claim 71, wherein the characteristic is an inductance of the sensing means.

73. The surgical instrument of claim 72, wherein the sensing means is configured for monitoring the proximity of a ferrous foreign body to the sensing means.
